(19) United States
(12) Patent Application Publication
(10) Pub. No.: US 2004/0023309 A1
(43) Pub. Date: Feb. 5, 2004

(54) Immunoassay and kit for an early and simultaneous detection of biochemical markers in a patient's sample

(76) Inventor: Franz Noll, Berlin (DE)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/304,552

(22) Filed: Nov. 26, 2002

Related U.S. Application Data
(60) Provisional application No. 60/333,133, filed on Nov. 27, 2001.

(51) Int. Cl. G01N 33/53

(52) U.S. Cl. 435/7.2

(57) ABSTRACT

The present invention comprises an immunochemical assay for determination of at least two antigens in a sample. The immunochemical assay comprises contacting a sample from a patient with a carrier molecule that contains at least two capture antibodies, each of which specifically binds to a binding moiety of an antigen in the sample. The assay further contains a detection antibody that specifically binds the same antigens on different binding moieties than binding moieties used by the capture antibodies. The detection agent is further attached to one or more detection probes to facilitate the detection of antigens in the sample. The immunochemical assay of the present invention is specifically designed to detect biochemical markers that are released at different time intervals in a patient's sample.

### RELATED APPLICATIONS

This application claims benefit of U.S. Provisional Application No. 60/333,133 filed Nov. 27, 2001. The aforementioned is hereby incorporated by reference in its entirety.

### FIELD OF THE INVENTION

The present invention relates to compositions and methods for detection of biochemical markers present in a biological sample at different time intervals after the onset of disease or disorders.

### BACKGROUND OF THE INVENTION

Delayed diagnosis of a disease often leads to a greater risk of permanent damage to tissues or even increased risk of mortality. In many diseases there are marker molecules which increase in expression in correlation with disease progression. Marker molecules are antigens associated with or produced by a disease, and may change in concentration concurrently with an increase in progression of the disease. Thus, the increase in the marker molecule may correlate with an increase in pathogenicity, and hence a worsening of the disease condition, i.e., a viral pathogen such as HIV, or a bacterial pathogen such as Salmonella. The diseased organism may also react to a pathogen or pathogenic condition, by producing or increasing production of markers that are not normally present or are only present in low levels in the organism, i.e., heart attack victims show increased levels of CK-MB, Troponin-T or I.

There are a number of biochemical markers available to detect or rule out ischaemic damage to the heart muscle cells caused by incidents such as acute myocardial infarction (AMI) or unstable Angina Pectoris with ST-T alterations. However, most of these markers are neither heart-specific nor are they detectable early enough after an AMI to be useful for early diagnosis.

The detection of creatine kinase MB isoenzyme (EC 2.7.3.2) in serum is currently the most widely utilized in vitro test for confirming the diagnosis of myocardial infarction (“heart attack”). However, while this test generally provides satisfactory results, there are some disadvantages which limit its utility. One disadvantage is the relatively short period (24-48 hours) the test remains positive following an infarction. In patients who arrive at the hospital more than 48 hours after onset of chest pain, the CK-MB test is generally not useful in confirming the diagnosis of a heart attack. In addition to this, skeletal muscle tissue normally contains small amounts of the CK-MB isoenzyme and therefore patients who suffer trauma to skeletal muscle tissue (i.e. in automobile accidents) will sometimes give false positive results making the diagnosis of myocardial infarction more difficult.

It is to the advantage of the diagnostician to identify markers as quickly as possible in order to initiate a proper therapeutic regimen to minimize the risk of mortality or morbidity. For most detection assays, there is a lag time for the markers to reach detectable concentrations. In the case of heart attacks, there is a delay of 4-6 hours from the onset of chest pain until there are detectable levels of CK-MB, troponin-T or troponin-I. Myoglobin is detectable earlier, but the current test has low specificity. Accordingly, there is a need for an assay which can rapidly detect minute levels of biochemical markers in a sample. There is also a need for an assay which can monitor the progression of a heart attack as well as the onset of successive infarcts.

### SUMMARY OF THE INVENTION

The present invention comprises an immunochemical assay for the detection of markers and the levels of the markers at different times following injury. The present invention further comprises an immunochemical assay for simultaneous determination of at least two antigens in a sample comprising contacting the sample with a carrier molecule comprising at least two capture agents which specifically bind to the binding moiety of at least one antigen. The immunochemical assay further comprises detection agents which bind to the antigens bound to the capture agents. The detection agents may additionally be coupled to a detection probe or the detection agents may be added separately to the sample. Alternatively, the carrier molecule also comprises a detection agent coupled to a detection probe and the detection agent specifically binds the antigens. The assay of the present invention can be used to detect heart specific markers glycogenphosphorylase BB (GPBB) and cardiac troponin-1.

The detection agents and capture agents of the assay may comprise antibodies or antibody fragments, including monoclonal, polyclonal, humanized, human, chimeric, recombinant, bispecific, multispecific antibodies, or a combination thereof. The antibody fragments may comprise Fab, Fab(2)′ Fe, Fv, single chain antibody, or a combination thereof.

The detection probe may be detectable enzymes, prosthetic groups, paramagnetic groups, fluorescent materials, luminescent materials, bioluminescent materials, radioactive materials, disperse dyes, gold particles, or a combination thereof.

A further embodiment of the present invention provides a composition that contains the carrier molecule as disclosed above, and additionally contains a carrier or diluent for internal consumption. The composition of this invention can be used diagnostically or therapeutically.

Another embodiment of the present invention provides a kit for detecting at least two biochemical markers of interest in a sample. The kit comprises a carrier molecule containing at least two capture agents, and further comprising at least one, preferably at least two detection agents. Each capture agent specifically binds to a binding moiety of one antigen and the detection agents bind to the same antigens via different binding moieties. The detection agent further comprises a detection probe that is either coupled to the detection agent or is provided separately to the sample.

### DETAILED DESCRIPTION OF THE INVENTION

The present invention comprises compositions and methods for detecting the occurrence of an acute myocardial infarction (AMI) or other damage to heart muscle, based upon testing for both early onset and late onset biochemical markers. The present invention further comprises immunochemical methods for the detection of proteins in a sample at different time points following the occurrence of an AMI in order to follow the progression of the AMI and determine if there have been successive infarcts. Testing for markers present at different times after onset of an AMI allows for early detection of ischaemic heart muscle damage leading to early treatment; specificity of diagnosis through the use of markers that are released by the heart and not other types of muscle; monitoring of successive AMIs; direct evaluation of the results and a measurement of the severity of the damage.

The present invention comprises an immunochemical assay for the determination of at least two biochemical markers in a sample comprising contacting the sample with a carrier molecule that contains at least two capture agents and at least one detection agent. Each capture agent specifically binds to a binding moiety of an antigen. The detection agent specifically binds the same antigen at different binding moieties than the binding moieties used by the capture agent. The detection agent further comprises a detection probe that is either coupled to the detection agent or is added separately to the sample. The immunochemical assay of the invention is capable of detecting simultaneously the presence and amount of two or more different antigens that are released at different time intervals in a patient's sample.

Definitions

The term “capture agent” as described herein includes any molecule, i.e., antibodies or antibody fragments, peptides or peptide fragments, enzymes, proteins, peptide complexes, peptide and carbohydrate complexes, nucleic acid molecules, or other chemical entities, so long as it has a binding specificity that binds to, or interacts with at least one antigen of interest.

The term “detection agent” includes any moiety, i.e., antibodies or antibody fragments, peptide or peptide fragments, enzymes, proteins, peptide complexes, peptide and carbohydrate complexes, nucleic acid molecules, or other chemical entities, so long as it has more than two different binding specificities which bind to, or interact with (a) at least one antigen of interest and (b) a binding moiety of at least one detection probe. Accordingly, the detection agent includes, but is not limited to, heteroantibodies, bispecific, trispecific, tetraspecific, and other multispecific molecules, which bind to an antigen of interest and to a detection probe. The detection agent may also be bound to the carrier molecule.

The term “detection probe” as used herein, refers to agents that are either coupled to the detection agent or to the sample containing the detection agent to facilitate identification of a complex molecule. The term “detection probe” includes probes, which are capable of interacting with the detection agent and forming detection agent and probe complexes. In one embodiment, the detection probe is labeled with 1 or more, 2 or more, 3-6, 6-12, 12-20, or more than 20 detectable labels. Examples of detectable labels include, but are not limited to, various enzymes, prosthetic groups, fluorescent materials, luminescent materials, bioluminescent materials, and radioactive materials.

The term “specific binding” as used herein includes antibodies that bind to one antigen with higher affinity than other related antigens. Typically, the antibody binds with an affinity of at least about 1×10⁷ M, and binds to the predetermined antigen with an affinity that is at least twice greater than its affinity for binding to a non-specific antigen (i.e., BSA, casein) other than the predetermined antigen or a closely-related antigen. For example, an antibody with specific binding affinity for troponin-I may bind troponin-I with higher affinity than troponin-T.

The phrases “an antibody recognizing an antigen” and “an antibody specific for an antigen” are used interchangeably herein with the term “an antibody which binds specifically to an antigen.”

The term “carrier molecule” as described herein includes a solid phase surface. The solid phase surface is not limited to any particular form. The solid surface can be selected from a variety of those known in the art including plastic tubes, beads, microtiter plates, latex particles, magnetic particles, cellulose beads, agarose beads, paper, dipsticks, and the like. More preferably, the carrier molecule of the invention comprises dipsticks.

The term “antigen” as used herein, includes any molecule or biochemical markers that can be detected using the immunoassay of the invention. The antigen includes, for example, any molecule that is newly expressed or exhibits increased or decreased expression in the body as a result of a disease or disorder. The term includes, for example, small molecules present in body fluids such as drugs, toxins, autoantibodies, autoantigens, proteins, carbohydrates, nucleic acids, or a combination thereof.

The term “sample” as used herein includes mixtures that contain the antigen. Preferably, samples are obtained from living sources, such as animals, i.e., mammals, and more preferably humans. The sample preferably is a body fluid, i.e., blood, plasma, saliva, urine, etc. and also includes tissue samples.

The term “antibodies or antibody fragments”, as used herein, refers to antibodies or fragments thereof that specifically bind to an antigen. Antibodies or fragments that specifically bind to a molecule can be identified, for example, by immunoassays or other techniques known to those of skill in the art.

The term “recombinant antibody” includes all antibodies that are prepared, expressed, created or isolated by recombinant means, such as phage display antibodies, antibodies isolated from a transgenic animal (i.e., a mouse), antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

The term “monoclonal antibody” includes antibodies which display a single binding specificity and affinity for a particular epitope. Preferably, these antibodies are mammalian antibodies, including murine, human and humanized antibodies.

The term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, i.e., a transgenic mouse, having a genome comprising a human heavy chain transgene and a light chain transgene fused to an immortalized cell.

The term “humanized antibodies” refers to antibody molecules from non-human species having one or more complementarity determining regions (CDRs) from the non-human species and a framework region from a human immunoglobulin molecule. (See i.e., U.S. Pat. No. 5,585,089, which is incorporated herein by reference in its entirety.) Such chimeric and humanized monoclonal antibodies can be produced by recombinant DNA techniques known in the art, for example using the methods described in U.S. Pat. Nos. 4,816,567 and 5,225,539, each of which incorporated herein by reference in its entirety.

“Chimeric antibodies”, according to the invention, are made recombinantly or chemically. For example, recombinant chimeric antibodies are made by splicing the genes from a monoclonal antibody of appropriate antigen specificity together with genes from a second human antibody of appropriate biological activity. More particularly, the chimeric antibody may be made by splicing the genes encoding the variable regions of an antibody together with the constant region genes from a second antibody molecule. This method is used in generating a humanized monoclonal antibody wherein the complementarity determining regions are mouse, and the framework regions are human (see, U.S. Pat. Nos. 4,816,567; 4,816,397; 5,693,762; 5,585,089; 5,565,332 and 5,821,337 each of which is incorporated herein by reference in its entirety).

As used herein the term “troponin” refers to a complex of troponin isoforms or individual troponin isoforms. There are nine troponin forms including: 1) the cardiac ternary complex; 2) the cardiac troponin binary complex of I(oxidized)/T; 3) the cardiac troponin binary complex of I(reduced)/T; 4) the cardiac troponin binary complex of I(oxidized)/C; 5) the cardiac troponin binary complex of I(reduced)/C; 6) the cardiac troponin binary complex T/C; 7) unbound cardiac troponin-I (oxidized); 8) unbound cardiac troponin-I (reduced); and, 9) unbound cardiac troponin-T. As used herein, unbound troponin is troponin that is not in a complex. A troponin complex can be binary or ternary.

A number of biochemical markers are available to detect or rule out ischaemic damage to heart muscle as in AMI or unstable Angina Pectoris with ST-T alterations. However, most of these markers are neither heart-specific nor are they detectable early enough to be useful. Examples of markers which could be used in the assays of the present invention include, but are not limited to, troponin, including complexes of troponin isoforms or individual troponin isoform (i.e., troponin-I , troponin-C, troponin-T), tropomyosin, actin, GPBB, drugs (i.e., barbiturates, tricyclic antidepressants, and Digitalis), tumor antigens (i.e., antigens associated with breast, prostate, brain, liver, kidney, colon, pancreatic, stomach, or lung cancer), viral antigens (i.e., antigens associated with or produced by HIV, influenza or other viruses), bacterial antigens, hormones (i.e., thyroid stimulating hormone (TSH), human growth hormones, progesterone, testosterone, human chorionic gonadotrophin (hCG)), plasma proteins (i.e., a fibrin degradation product (FDP), a C-reactive protein (CRP), a carcinoembryonic protein, alpha-fetoprotein (AFP), carcinoembryonic antigen (CEA)), plaque antigens, haptens (i.e., angiotensin I, vasopressin, somatostatin, atrial natriuretic hormone, endoserine, luteinizing hormone releasing hormone (LH-RH), kassinin or other peptides), steroids (i.e., cortisol), and cytokines such as interleukin-1 (IL-1), interferon-alpha, interferon-beta, interferon-gamma, interleukin-2 (IL-2), interleukin-4 (IL-4), interleukin-6 (IL-6), interleukin-7 (IL-7), interleukin-12 (IL-12), interleukin-15 (IL-15), B7, CD28, or other members of the Ig superfamily.

The present invention further comprises the use of the BB iso-enzyme of glycogenphosphorylase as an early onset antigen for heart muscle damage. While not wishing to be bound by any particular theory, glycogen phosphorylase isoenzyme BB (GPBB) is reported to be a key enzyme for the early detection of ischaemic heart disorder (Mair J., Clin Chim Acta 6; 272:79-86 (1998)). Considerable amounts of GPBB are only found in human heart and brain. GPBB is detectable in the blood within 2 hours of the beginning of chest pains. GPBB levels also increase early in patients with unstable angina and reversible ST-T alterations in the resting electrocardiogram at hospital admission, which could be useful for risk stratification. GPBB is heart-specific and it disappears from blood 16-24 hours after the appearance of the ischaemic damage to the heart muscle. The short life-time of the enzyme makes it useful as an indicator of renewed infarction, after by-pass surgery for example, simply by detecting enhanced levels of GPBB in the blood stream.

The present invention additionally comprises immunochemical assays and methods of diagnosis comprising the pairing of GPBB as an early onset marker with a late onset marker, specific for ischaemia-induced damage in heart muscle. This late onset marker is preferably cardiac troponin-I. Cardiac troponins in serum are structural proteins of the heart muscle cells and, as such, its release is a sign of beginning necrosis in the heart muscle. The late marker, cardiac troponin-I, appears in circulation about 4 hours after the inception of chest pain; its life-time in circulation is significantly longer than that of GPBB. Measurement of cardiac troponin-I therefore allows the diagnosis of an ischaemic event many hours or days after it has occurred.

The present invention comprises compositions and methods of detection assays with increased specificity. This is achieved by the use of carrier molecules that bind to at least two antigens that are present in a patient's sample at different time intervals after the onset of a disease.

Specifically, the carrier molecule comprises at least two capture agents, and may additionally comprise at least one detection agent. Each capture agent binds to a different antigen or marker. The detection agent may be part of the carrier or added separately. The detection agent specifically binds the antigens bound to the capture agents at different binding moieties. The detection agent further comprises a detection probe that is either coupled to the detection agent or is added separately to the sample.

The present invention further comprises methods for determing the presence of at least two antigens in a sample comprising contacting a sample from a human or animal with a carrier molecule comprising at least two capture agents, each having binding specificity for different antigens, to form a reaction mixture. A detection agent is then added to the reaction mixture and a detection probe may be bound to the detection agent or added to the reaction mixture. The concentration of the at least two antigens in the sample is then determined.

The immunochemical assay of the invention is preferably used to detect the onset of myocardial infarction. According to one aspect, the present invention features a method for detecting myocardial infarction, comprising the steps of contacting a patient's sample with a carrier molecule that contains two capture agents to form a reaction mixture. One capture agent binds to a binding moiety of GPBB and the other capture agent binds to a binding moiety of troponin-I. The detection agents are added to the reaction mixture, wherein the detection agents comprise a reagent, preferably monoclonal antibodies specific for GPBB and troponin-I, whereby their binding sites differ from binding sites of the capture antibodies fixed to a solid phase. The detection agents additionally bind detection probes. The immunoassay of the invention is capable of detecting both troponin-I and GPBB simultaneously. A further embodiment comprises a carrier molecule with capture agents and detection agents.

According to another embodiment of the invention, samples are taken from the patient at different times, for example, following a suspected myocardial infarction, and tested in the assay in order to follow the progression of the disease or to detect subsequent myocardial infarctions.

A further embodiment of the invention comprises a method for detection of myocardial infarction in a patient, comprising: contacting a sample from the patient with a carrier molecule, the carrier molecule comprising an anti-GPBB monoclonal antibody, an anti-cardiac troponin-I monoclonal antibody, and at least one detection agent, and detecting the concentration of GPBB and cardiac troponin-I in the sample wherein the detection agent binds both the GPBB and troponin-I at binding moieties that are not used by the capture agents. The assay can be repeated with new samples over an extended period time to monitor the progress of the injury and to detect any subsequent infarcts.

According to one embodiment of the invention, the capture agent, the detection agent, or both are antibodies or antibody fragments. In a preferred embodiment of the invention, antibodies are monospecific, bispecific, or multispecific antibodies. Methods for preparing bi- and multispecific molecules are described, for example, in U.S. Pat. Nos. 5,260,203; 5,455,030; 4,881,175; 5,132,405; 5,091,513; 5,476,786; and 5,013,653, each of which is incorporated herein by reference in its entirety.

In a preferred embodiment, the bispecific or multispecific antibody has a first variable region having specificity to a molecule to be detected and a second variable region having specificity for a second molecule. The antibody is linked to a polymer, wherein the polymer is attached to at least 1, or more, preferably 2 or more detection molecules.

In particular, detection antibodies comprise at least two binding regions specific for two antigens of interest to be detected and another binding region specific for a probe which is added separately.

According to one embodiment of the invention, the antibody moieties are linked together to form an antibody conjugate. Antibody conjugates include heteroantibodies, which refer to two or more antibodies or antibody fragments linked together, wherein the antibody conjugate has at least two binding regions with different specificities. These different specificities may advantageously include, for example, a binding specificity for the binding moiety of the detection probe, and two binding specificities for two antigens of interest, i.e., GPBB and cardiac troponin antigens.

According to another embodiment of the invention, antibodies or antibody fragments are made recombinantly. In a preferred embodiment of the invention, antibodies or antibody fragments are monoclonal antibodies. In another embodiment, antibodies or antibody fragments are produced by isolation of the individual monoclonal antibodies, breaking of disulfide linkages of each specific antibody and subsequent recombination of antibody heavy and light chain polypeptides in vitro (see, for example, Arathoon et al., WO 98/50431). In yet another embodiment, the invention uses one or more chimeric antibodies in the immunochemical assay.

The antibodies of the invention, include immunologically active fragments of immunoglobulin molecules, i.e., F(ab) and F(ab′)2 fragments, which can be generated by treating the antibody with an enzyme such as pepsin or papain. Examples of methods of generating and expressing immunologically active fragments of antibodies can be found in U.S. Pat. No. 5,648,237, which is incorporated herein by reference in its entirety.

The immunoglobulin molecules are encoded by genes which include the kappa, lambda, alpha, gamma, delta, epsilon and mu constant regions, as well as any number of immunoglobulin variable regions. Light chains are classified as either kappa or lambda. Light chains comprise a variable light (VL) and a constant light (CL) domain. Heavy chains are classified as gamma, mu, alpha, delta, or epsilon, which in turn define the immunoglobulin classes IgG, IgM, IgA, IgD and IgE, respectively. Heavy chains comprise variable heavy (VH), constant heavy 1 (CH1), hinge, constant heavy 2 (CH2), and constant heavy 3 (CH3) domains. The human IgG heavy chains are further sub-classified based on their sequence variation, and the subclasses are designated IgG1, IgG2, IgG3 and IgG4.

Antibodies can be further broken down into two pairs of a light and heavy domain. The paired VL and VH domains each comprise a series of seven subdomains: framework region 1 (FR1), complementarity determining region 1 (CDR1), framework region 2 (FR2), complementarity determining region 2 (CDR2), framework region 3 (FR3), complementarity determining region 3 (CDR3), framework region 4 (FR4) which constitute the antibody-antigen recognition domain.

In another embodiment, the invention uses a single-chain antibody (scFv), which generally comprises a fusion polypeptide consisting of a variable domain of a light chain fused via a polypeptide linker to the variable domain of a heavy chain.

Detection can be facilitated by coupling the antibodies to detectable labels. Examples of detectable labels include, but are not limited to various enzymes, prosthetic groups, fluorescent materials, luminescent materials, bioluminescent materials, radioactive materials, disperse dyes, and gold particles. Examples of suitable detectable labels, as disclosed above, include suitable enzymes, i.e., horseradish peroxidase, alkaline phosphatase, betagalactosidase, or acetylcholinesterase; examples of suitable prosthetic group complexes include, but are not limited to streptavidin/biotin and avidin/biotin; examples of suitable fluorescent materials include, but are not limited to umbelliferone, fluorescein, fluorescein isothiocyanate, rhodamine, dichlorotriazinylamine fluorescein, dansyl chloride or phycoerythrin; an example of a luminescent material includes, but is not limited to luminol; examples of bioluminescent materials include, but are not limited to luciferase, luciferin, and aequorin; and examples of suitable radioactive material include, but are not limited to 125I, 35S, 14C, 3H, Tc99M, or Mg52. The antibodies can be coupled to the same or different detection labels.

Antibodies that are commercially available can be purchased and used to generate the detection agent, i.e., from ATCC.RTM. In a preferred embodiment of the invention, the antibody is produced by a commercially available hybridoma cell line. In a more preferred embodiment, the hybridoma secretes a human antibody.

All previously existing ELISA, radioimmunoassays, and dipstick assays for detection of serum enzymes, and any assays utilizing antibodies could be modified according to the method of the invention to provide enhanced sensitivity. In addition, in vivo application to enhance the target signal by using the method of the invention is also possible.

The capture agent of the invention can be directly affixed to the solid phase surface, or can be immobilized during the immunoassay incubation (in situ) by means known to those skilled in the art. The capture agents are preferably immobilized on the surface of the carrier molecule. The methods for immobilizing the capture agents on the surface of the carrier molecule are not limited to any particular method and include, for example, passive absorption, covalent linkage, physical trapping, and the like. For example, the solid phase surface can be coated and/or the detection agent can be labeled with avidin or streptavidin.

Alternatively, the capture agent or detection agent can be added in liquid phase to the biological fluid containing the antigens of interest, although as indicated above, the capture agents and the detection agent are preferably fixed on the surface of the carrier molecule.

According to one embodiment of the invention, the antibody has specific binding affinity for one or more proteins enumerated herein. For example, an antibody has specific binding affinity for only troponin-I and not troponin-T. Alternatively, an antibody may specifically bind to both troponin-I and troponin-T. An antibody has specific binding affinity for two or more proteins because, for example, (a) the antibody binds to discrete epitopes that are conserved in the two proteins, or (b) the antibody binds to separate and adjacent epitopes on two proteins. In example (a), the antibody may bind to proteins separately, however in example (b), the antibody may bind to proteins when they are in complex with one another.

The form of troponin released by the heart, whether free or as binary or ternary complexes, may indicate a particular condition of the heart. The assays described herein provide for the analysis of release patterns of markers which allow the physician to diagnose the patient's condition, for example, unstable angina as compared to myocardial infarction or to determine the time that an infarction occurred.

According to another embodiment of the invention, the carrier molecule contains an antibody specific for troponin-I or troponin-T, and a second antibody that is specific for GPBB and further attached to one or more detection probes. Troponin-I is one of three subunits of the troponin complex located on the thin filament of the muscle contractile apparatus. This troponin complex plays a central role in controlling the process of muscle contraction, and therefore these three subunits are called regulatory proteins. The other two subunits (designated T and C) are also immobilized on the thin myofilaments along with troponin-I in both cardiac and skeletal muscle tissue. Troponin-I is encoded by different genes in cardiac, slow skeletal, and fast skeletal muscle tissues. Approximately 60% of the amino acid sequence in humans is homologous between these three forms of troponin. The dissimilar regions of the cardiac form make it possible to develop antibodies which will not cross react with the two skeletal forms, thus making a cardiac specific test possible.

Cummins, et al., American Heart Journal 113:1333-1344 (1987) described the development of a radioimmunoassay for the measurement of cardiac troponin-I in human serum. This assay utilized polyclonal antibodies having significant cross reactivity with the skeletal forms of troponin-I, which limited its value in confirming the diagnosis of myocardial infarction. In addition, the test was not sufficiently sensitive to detect low levels of troponin-I in serum.

Bodar, et al., Clinical Chemistry 38:2203-2214 (1992) described the development of a dual monoclonal antibody “sandwich” assay for troponin-I in serum. While this assay showed improved cardiac specificity due to the use of mouse monoclonal antibodies, the imprecision of the assay was unacceptably high (11-21% coefficients of variation) for a laboratory test.

According to one aspect of the invention, there is provided a kit for detecting at least two biochemical markers of interest in a sample. The kit comprises a carrier molecule containing at least two capture agents, and at least one detection agent, each capture agent specifically binds to a binding moiety of one antigen and the detection agent binds to the same antigens via different binding moieties. The detection agent further comprises a detection probe that is either coupled to the detection agent or is provided separately to the sample.

According to another aspect of the invention, a diagnostic or therapeutic composition is disclosed that is capable of binding to at least two biochemical markers in vitro or in vivo. Specifically, the composition contains a carrier molecule having at least two capture agents with binding specificity to at least two antigens, further comprising a detection agent having binding specificity to the same antigens, and further comprising a detection probe. In one embodiment, the detection agent is a recombinantly expressed bispecific antibody raised against troponin-I and GPBB. In another embodiment, the detection agent comprises two monoclonal antibodies raised against troponin and GPBB, respectively.

As used herein and in the appended claims, the singular forms “a,” “an,” and “the” include plural reference unless the context clearly indicates otherwise. Thus, for example, reference to a “compound” is a reference to one or more such compounds and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are incorporated herein by reference for the purpose of describing and disclosing, for example, the constructs and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

It is to be understood that this invention is not limited to the particular formulations, process steps, and materials disclosed herein as such formulations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

### EXAMPLES

### Example 1

Immunochemical Assay for Diagnosis of Ischaemic Heart.

This immunochemical assay is based on the use of a solid phase carrier molecule containing a two-site-binding assay using two different monoclonal antibodies (mAb), one against the early onset marker GPBB, and the other against the late onset marker troponin-I. The two mAbs are adsorbed at different sites on the solid phase. The adsorbed antibodies act as “capture antibodies” which specifically bind the markers present in the patient's blood. A further antibody raised against a different epitope on the early onset and on the late onset marker is labeled with an enzyme or fluorescence dye or a dispersing dye or with gold particles. This antibody serves as a “detection antibody” for the antigens attached to site 1 and 2 of the solid phase. Both the capture mAb's against the early onset and the late onset marker bind at different epitopes on the markers thus yielding a 2-sites binding test. The data shows that an early stage of an acute myocardial infarction (AMI), i.e., 2-4 hours after onset of chest pains/behind the sternum is measurable at site 1 on the solid phase. The peak of an AMI (4-16 hours after onset of chest pains) is measurable at sites 1 and 2 and the late stage of an AMI (>24 hours) is measurable at site 2 only.

### Example 2

Purification of Troponin-I.

In order to produce mouse anti-troponin-I antibodies, cardiac troponin-I is first isolated by the method of Syska et al., FEBS Letters 40:253-257(1974) as follows. Approximately 500 mg of troponin-I is coupled to ACTIGEL-ALD gel (Sterogene Corporation, Arcadia, Calif.) by washing 50 ml of the gel with 10 mM potassium phosphate, 1M potassium chloride, pH 6.5 (coupling buffer). Troponin-C is then added to the gel and sodium cyanoborohydride is added to a final concentration of 0.1M. The resulting suspension is allowed to stir for four hours at ambient temperature and poured into a column to collect the gel. The gel is then washed with 225 ml of coupling buffer. The gel is removed from the column and is added to 150 ml of 10 mM potassium phosphate, 1M potassium chloride pH 6.5 containing 0.1M ethanolamine. Sodium cyanoborohydride is added to the suspension to a final concentration of 0.1M. The suspension is allowed to stir overnight at 4° C. to block any unreacted coupling groups. The gel is then placed back in a column and washed with 150 ml of coupling buffer, and finally with 100 ml of 10 mM sodium phosphate pH 7.2 containing 0.15M sodium chloride and 0.05% sodium azide.

A human heart is trimmed and cut into 1 cm pieces at 4° C. The resulting tissue is homogenized with 750 ml of 75 mM Tris buffer, pH 8.0 containing 8M urea, 15 mM mercaptoethanol and 1 mM calcium chloride (extraction buffer) at ambient temperature.

The resulting homogenate is centrifuged for 30 minutes at 7000× g and the resulting supernatant liquid is filtered through cheesecloth to remove particles. The troponin-C coupled gel prepared above is placed in a column and washed with 250 ml of extraction buffer at ambient temperature. The gel is removed from the column and added to the filtered heart extract. The resulting suspension is allowed to stir for 80 minutes at ambient temperature and then centrifuged for 20 minutes at 7000× g. The supernatant liquid is discarded and the pelleted gel is transferred to a column with extraction buffer. The column is washed at ambient temperature with a total of 700 ml of extraction buffer and the purified troponin-I is then eluted from the column with 75 mM Tris buffer, pH 8.0 containing 8M urea, 15 mM mercaptoethanol, and 10 mM ethylenediamine tetraacetic acid (elution buffer). Fractions containing significant amounts of troponin-I are pooled together and added to 75 mM Tris buffer, pH 8.0 containing 10 mM ethylenediamine tetraacetic acid and 15 mM mercaptoethanol. The resulting solution is concentrated under nitrogen pressure.

### Example 3

Preparation of Mouse Anti-Troponin-I Antibodies.

The purified troponin-I, obtained from the procedure of Example 2 above, is mixed with an equal volume of complete Freunds adjuvant. The resulting mixture is homogenized to produce an aqueous/oil emulsion which constitutes the initial immunogen. Mice are immunized initially with an injection of immunogen containing 250 μg of cardiac troponin-I. Mice are injected monthly thereafter with 250 μg-500 μg of purified cardiac troponin-I as immunogen, then they are bled monthly approximately 7-10 days after injection to provide mouse anti-troponin-I serum.

### Example 4

Isolation and Purification of Cardiac Specific Troponin-I Antibodies.

The antiserum prepared in Example 3 is collected and 56 ml of it is diluted with 56 ml of 5 mM imidazole buffer pH 7.2 containing 0.15M sodium chloride. Phenylmethyl sulfonyl fluoride (PMSF), leupeptin, aprotinin, and pepstatin A are added to final concentrations of 15 μg/ml, 0.5 μg/ml, 0.5 μg/ml and 0.75 g/ml respectively in order to inhibit proteases in the antiserum. The synthetic peptide gel prepared in Example 2 is added to the diluted antiserum and allowed to stir for 1 hour at ambient temperature. The resulting mixture is transferred to a column and washed with 55 ml of 5 mM imidazole pH 7.2 containing 1M sodium chloride and 0.05% sodium azide at ambient temperature. The purified cardiac specific antibodies are eluted from the gel with 55 ml of first elution buffer, followed by 55 ml of second elution buffer (5 mM imidazole pH 7.0 containing 3M sodium thiocyanate and 0.05% sodium azide). The purified antibodies contained in both these eluates are dialyzed to a final dilution of 10⁶ against 5 mM imidazole pH 7.2 containing 0.15M sodium chloride, concentrated under nitrogen pressure to approximately 25 ml and then dialyzed to a final dilution of 10⁹ in 10 mM sodium phosphate pH 7.2 containing 0.15M sodium chloride and 0.05% sodium azide. The resulting dialyzate is then centrifuged for 15 minutes at 7000× g to remove insoluble material. The protein concentration of the resulting supernatant liquid containing purified cardiac-specific troponin-I antibodies is determined spectrophotometrically.

### Example 5

Preparation of Troponin-I-Alkaline Phosphatase-Conjugate.

Troponin-I prepared by the method of Example 3 is chemically linked to alkaline phosphatase by the following procedure. Troponin-I is treated with 25 μl of SATA (N-succinimidyl S-Acetylthioacetate). After allowing the reaction solution to stir for 30 minutes at room temperature, the solution is dialyzed overnight against 2 liters of 50 mM sodium phosphate pH 7.5 containing 2 mM EDTA at 4° C. The SATA modified troponin-I is deacetylated by adding hydroxylamine to a final concentration of 50 mM and allowing the solution to stand at ambient temperature for two hours. The modified troponin-I is then dialyzed overnight against 2 liters of 30 mM triethanolamine pH 7.2 containing 2 mM EDTA. Six mg of alkaline phosphatase (AP), from calf intestine (Biozyme Corporation, San Diego, Calif.) in a volume of 1.55 ml, is placed in a glass test tube. A fresh solution of sulfo-SMCC (sulfosuccinimidyl 4-N-maleimidomethyl! cyclohexane-1-carboxylate) is prepared at a concentration of 5 mg/ml in deionized water. A total of 87 μl of the SMCC solution is added to the AP and allowed to stir for one hour at ambient temperature.

The modified AP solution is then dialyzed overnight against 2 liters of 30 mM triethanolamine pH 7.2 containing 5 mM magnesium chloride and 1 mM zinc chloride at 4° C. A total of 1.35 mg of the SATA modified troponin-I is mixed with 4 mg of SMCC modified AP and allowed to stir for 24 hours at 4° C. Mercaptoethylamine and iodoacetamide are added to the solution to a final concentration of 10 mM and allowed to stir for 20 minutes at ambient temperature. The resulting AP conjugated troponin-I is then passed over a column of SEPHACRYL S-300 (Pharmacia Biotech Inc., Piscataway, N.J.) to purify the AP troponin-I conjugate from unreacted products.

### Example 6

Troponin-I Immunoassay Competitive Binding.

Purified troponin-I antibodies prepared according to Example 4 are diluted to 10 μg/ml in 100 mM sodium citrate pH 4.0 containing 0.05% sodium azide. The antibodies are coated overnight at ambient temperature in a volume of 100 μl to polystyrene microtiter plates. The microtiter plates are washed three times with 10 mM Tris buffer pH 7.2 containing 1M sodium chloride and blocked with a solution containing 10 mM Tris pH 7.2, 10% gluconic acid, 1% bovine serum albumin and 0.05% PROCLIN™. 300 5-chloro-2-methyl-4-isothiaxolin-3-one (CAS 26172-55-4), 2-methyl-4-isothiazolin-3-one (CAS 2682-20-4), alkyl carboxylate, modified glycol. Excess liquid is aspirated from the microtiter plate wells and the plates are allowed to dry at ambient temperature. The antibody coated plates are then stored at 4° C. until use. Purified cardiac troponin-I prepared as in Example 2 is diluted in troponin-I-free normal human serum to final concentrations of 5, 25 and 50 ng/ml to provide standards for the immunoassay. Troponin-I labelled alkaline phosphatase is diluted to 5 μg/ml concentration in 50 mM triethanolamine pH 7.4, 1 mM magnesium chloride, 0.1 mM zinc chloride and 0.05% sodium azide.

Serum samples or troponin-I standards are added in duplicate to the antibody coated microtiter plate wells prepared previously. Troponin-I labelled AP (80 μl) is then added to the wells and incubated for two hours at ambient temperature. The microtiter plate wells are then washed five times with deionized water and a substrate solution (100 μl) of 0.83 mg/ml paranitrophenyl phosphate in 25 mM diethanolamine pH 9.80 containing 5 mM magnesium chloride, 0.1 mM zinc chloride, 0.02% TWEEN 20, and 0.05% PROCLIN 300 is then added to all of the wells. The substrate solution is allowed to incubate for 30 minutes at ambient temperature and the reaction is stopped by the addition of 100 μl of 2 N sodium hydroxide. Absorbance of the solutions in the microtiter plates are then read at 405 nm with a suitable reader.

### Example 7

Preparation of Biotinylated Troponin Antibodies Avidin-HS Magnetic Latex.

Biotin-succinimidyl ester (6-((6-((biotinoyl)amino)hexanoyl)amino) hexanoic acid, succinimidyl ester, at 40 mM in dimethylformamide is added slowly with mixing to an antibody solution at 2 mg/ml in 50 mM potassium borate, 150 mM sodium chloride, pH 8.2, (BBS) to achieve a final molar ratio of 20/1 biotin-/antibody. The solution is incubated at room temperature for 2 h, after which the solution is dialyzed at 4° C. for at least 12 h.

One ml of Estapor Paramagnetic latex particles at 10% solids in water is added to 9 ml of 0.55 mg/ml avidin-HS (Scripps Laboratories, San Diego, Calif.) in 50 mM Tris hydrochloride, 150 mM sodium chloride, pH 7.5. The latex solution is incubated at 45° C. for 2 h. The latex is washed 3 times, each with 10 ml BBS, and resuspended in 10 ml BBS.

### Example 8

Immunoassay of Human Cardiac Troponin-I and Troponin-T.

The following immunoassay is used to detect troponin-I and troponin-T, present in human serum, plasma, or in solutions containing purified proteins.

The sample containing troponin-I or troponin-T is diluted to 1-10 ng/mil troponin-I or troponin-T in an assay buffer containing 10 mM 3-(N-morpholino) propane sulfonic acid, 650 mM sodium chloride, 1 mM magnesium chloride, 0.1 mM zinc chloride, 1 mg/ml polyvinyl alcohol (10,000 mw), 10 mg/ml bovine serum albumin, 1 mg/ml sodium azide, pH 7.0. To 25 microliter of diluted sample in a microtiter plate well is added 50 microliter of assay buffer containing 2.5 microgram/ml anti-troponin-I or anti-troponin-T antibody conjugates and 2.5 microgram/ml biotinylated anti-troponin-I or anti-troponin-T polyclonal antibody to form a reaction mixture. After a 30 minute incubation of the reaction mixture at room temperature, 25 microliters of avidin-HS coated magnetic latex (0.5% latex in assay buffer) is added to the microtiter plate well, followed by a 5 minute incubation at room temperature.

The magnetic latex is pelleted and washed twice in BBS-Tween (20 mM borate, 150 mM sodium chloride, 0.1 mg/ml sodium azide, 0.02% Polyoxyethylene-20-Sorbitan Monolaurate (Tween-20), pH 8.2) and once in TBS (40 mM Tris, 150 mM sodium chloride, pH 7.5). The pellet is resuspended in ELISA amplification reagents (Gibco BRL, Gaithersburg, Md.) according to the manufacturer's instructions. After the amplification is complete, the magnetic latex is pelleted and 80 microliters of the colored supernatant is transferred to a fresh microtiter plate. The absorbance at 490 μm is measured using a microtiter plate reader.

Many modifications and variations of the present invention are possible in light of the above teachings. It is, therefore, to be understood within the scope of the appended claims the invention may be protected otherwise than as specifically described.

What is claimed is:

1. A method of determining the presence of at least two antigens in a sample comprising:
a) contacting a sample from a human or animal with a carrier molecule comprising at least two capture agents, each having binding specificity for different antigens, to form a reaction mixture.
b) adding at least one detection agent to the reaction mixture; and
detecting a concentration of the at least two antigens in the sample.

2. The method of claim 1, wherein the two antigens are heart specific early onset and late onset antigens, respectively.

3. The method of claim 2, wherein the early onset antigen is a glycogenphosphorylase BB (GPBB) and the late onset antigen is a cardiac troponin-I.

4. The method of claim 1, wherein the detection agents and the capture agent comprise antibodies or antibody fragments.

5. The method of claim 4, wherein the antibodies comprise, monoclonal, polyclonal, humanized, human, chimeric, recombinant, bispecific, multispecific antibodies, or a combination thereof.

6. The method of claim 4, wherein the antibody fragments comprise Fab, Fab(2)′ Fc, Fv, single chain antibody, or a combination thereof.

7. The method of claim 1, wherein the sample is from a human.

8. The method of claim 1, wherein the sample comprises a tissue, blood, saliva, plasma sample, lymphoid fluid, cerebrospinal fluid, or serum.

9. The method of claim 1, wherein the assay is conducted in vitro.

10. The method of claim 1, wherein the method further comprises a detection probe.

11. The method of claim 10, wherein the detection probe comprises, detectable enzymes, prosthetic groups, paramagnetic groups, fluorescent materials, luminescent materials, bioluminescent materials, radioactive materials, disperse dyes, gold particles, or a combination thereof.

12. A diagnostic test kit for early detection of acute myocardial infarction in a patient, comprising a carrier molecule containing at least two capture agents and further comprising at least one detection agent, each capture agent specifically binds to a binding moiety of an antigen, and the detection agent binds to the antigens via binding moieties that are not used by the capture agents.

13. The kit of claim 12, wherein the sample is a patient's tissue, blood, saliva, plasma, serum, lymphoid fluid, or cerebrospinal fluid.

14. The kit of claim 13, wherein the patient is a human.

15. The kit of claim 12, wherein the biochemical markers are GPBB and troponin-I.

16. The kit of claim 12, wherein the detection agent further comprises a detection probe.

17. The kit of claim 16, wherein the detection probe is coupled to the detection agent.

18. The kit of claim 16, wherein, the detection probe is not coupled to the detection agent.

19. A method for detection of myocardial infarction in a patient, comprising:
a) contacting a sample from the patient with a carrier molecule, the carrier molecule comprising an anti-GPBB monoclonal antibody, an anti-cardiac troponin-I monoclonal antibody, and at least one detection agent,
b)detecting the concentration of GPBB and cardiac troponin-I in the sample wherein the detection agent binds both the GPBB and troponin-I at binding moieties that are not used by the capture agents; and
c)repeating a) and b) with a new sample for a predetermined time period.

20. The method of claim 19, wherein the detection agent comprises one or more detection probes.
